(19) United States
(12) Patent Application Publication
(10) Pub. No.: US 2011/0261583 A1
(43) Pub. Date: Oct. 27, 2011

(54) LIGHT SOURCE

(76) Inventors: Volker Geuder, Heidelberg (DE); René Draheim, Sandhausen (DE)

(21) Appl. No.: 13/128,154

(22) PCT Filed: Oct. 16, 2009
(86) PCT No.: PCT/DE2009/001443

§ 371 Date: Jul. 18, 2011

(30) Foreign Application Priority Data
Nov. 10, 2008 (DE) 10 2008 056 565.2

(51) Int. Cl. A61B 1/06; H01L 33/02

(52) U.S. Cl. 362/555; 362/572

(57) ABSTRACT

A light source for coupling light into a medical device, particularly a surgical handheld device, preferably for use in ophthalmology, comprising a lamp (1), a light guide (2) extending from the lamp (1), and a power supply (3) for the lamp (1), is characterized in that the lamp (1) is arranged in an adapter-like housing (4), which comprises a connection (5) for the power sup ply (3) and a connection (6) for the light guide (2).

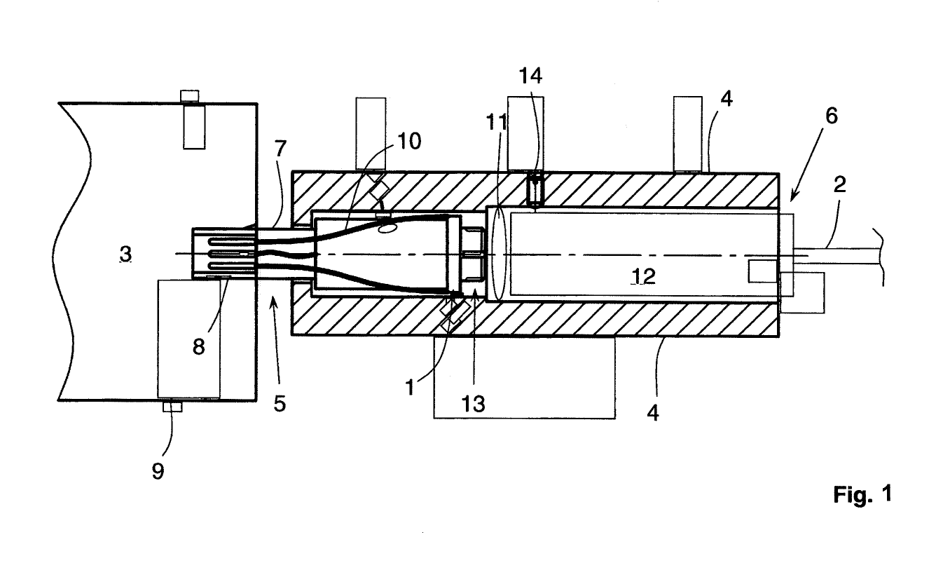

The invention relates to a light source for coupling light into a medical device, more particularly a surgical handheld device, preferably for use in ophthalmology, which light source comprises a lamp, a light guide extending from the lamp, and a power supply unit for the lamp.

Light sources for coupling light into a medical device, more particularly a surgical handheld device, are sufficiently known from practical experience. Thus light sources of this type are used, for example, in ophthalmology, where usually a stationary light source is provided, the light emitted from which is coupled into a handheld device by means of an optical fiber in order to illuminate an operating area on/in the eye. Such a light source comprises a lamp that can be of any desired type. As mentioned above, a light guide is provided that extends from the lamp to the location at which the light is required. Furthermore, a mostly integrated power supply unit is provided for the lamp in the prior art, where the light source together with the power supply unit forms a structural unit.

Light sources of the generic kind offer less flexibility since they are usually floor-mounted appliances that usually have to be connected to a stationary power supply system. Furthermore, the light sources of the generic kind require considerable space and are also less flexible in this respect. When illuminating light of different wavelengths is required, it is necessary to use expensive filters or replace the light source. This necessitates considerable design outlay and is cumbersome in terms of handling.

The object underlying the present invention is therefore to design and develop a light source for coupling light into a medical device, more particularly a surgical handheld device, preferably for use in ophthalmology in such a way that it has the smallest design and can be used flexibly. The light source is intended to be extremely simple in terms of handling.

The object mentioned above is achieved according to the invention by a light source having the features defined in claim 1. Accordingly, the generic light source is characterized in that the lamp is disposed in an adapter-like housing comprising a connection for the power supply unit and a connection for the light guide.

One finding according to the invention is that it makes sense to deviate from the hitherto customary design of a lamp together with the power supply unit in a common housing. Rather, the idea underlying the invention is to arrange the lamp in an adapter-like housing that can be handled as a structural unit independently of the power supply unit. For this purpose, the lamp is disposed in an adapter-like housing and it comprises two connections, namely a connection for the power supply unit and a connection for the light guide.

The light source of the invention is designed by means of the lamp disposed in the adapter-like housing in such a way that the lamp or the adapter can be replaced arbitrarily. Likewise, a mobile power supply unit can be connected to the adapter. Similarly, the adapter serves for coupling a light guide that then is in turn connected directly to a medical handheld device or the like.

In principle, it is also feasible for the adapter comprising the lamp to be directly flange-mounted on the handheld device. The power supply unit can be disposed in a separate housing on the rear side of the adapter. It is likewise feasible for the adapter to be coupled to the housing of a mobile power supply unit and for a light guide to extend between the adapter and the handheld device. Both variants are basically feasible and are covered by the idea of the invention.

Particularly, the housing forming the adapter can be designed together with the lamp located therein as a one-piece component. The housing can have any desired shape, but is preferably in the form of a cylindrical sleeve.

It is further advantageous if the connections for the power supply unit and the light guide respectively are disposed on opposite sides of the housing. A connection for the power supply unit can preferably be in the form of a plug assigned to the housing, which plug protrudes from an end side of the housing or the adapter. The plug can be a commercially available weak-current plug for power input or for the electrical connection to the power supply unit. In any case, it is advantageous if the plug protrudes at least slightly from an end side of the housing so that a plug-and-socket connection is possible, for example, to a storage battery pack. For preventing any accidental damage, a protective cap can be provided for the plug. It is also feasible to provide a sleeve that is disposed around the adapter housing in the region of the plug protruding from the housing. This sleeve can be pushed back, for example, against the force of a spring, and this process can take place virtually automatically when inserting the plug into a corresponding socket of the power supply unit.

It is important that the connection for the light guide be disposed directly close to the end side of the lamp. For this purpose, this connection could be in the form of a receptacle that is integrated in the housing and is intended for the coupling end of a light guide or a bundle of light guides. In the interior of the housing or the adapter, a stop could be provided in the form of an inner shoulder, and the coupling end of the light guide/bundle of light guides can be pushed close to this stop. The light source disposed in the housing or an optical system acting in between is connected immediately thereafter.

In any case, it is essential that the receptacle that is formed in the housing and is intended for the light guide or the bundle of light guides be defined substantially by the inner wall of the housing.

For preventing the light guide from slipping out accidentally, it is advantageous if means are provided for locking the light guide in its coupled position. These means can be snap-on or clamping means of any kind. In terms of construction, it is very simple to provide a screw, for example, a set screw as the clamping means, which set screw can be actuated from the exterior of the housing and can be screwed or pressed at least slightly against the coupling end of the light guide. It is thus ensured that the light guide is secured inside the adapter. Any other connections, for example, bayonet connections, can also be realized.

The housing or the adapter can be variably large corresponding to the lamp. Particularly with a view to miniaturize the adapter, it is very advantageous if the lamp is an LED or an array comprising a plurality of LEDs. It is also possible to integrate RGB LEDs having a corresponding control of the color selection. In principle, it is feasible to provide the adapter with LEDs of different wavelengths in the emission range.

As mentioned above, a lens or a lens system, a filter or other optically acting means can be disposed between the lamp and the coupling point for the light guide inside the housing. It is also feasible to provide the adapter with different optical means or to design the optical means so as to be replaceable by means of a small slot, if required. In any case, it is essential that the optical means be disposed downstream of the lamp and that these optical means control the light to be coupled into the light guide.

In the case of the smallest possible design of the light source, it is advantageous if a heat sink is assigned to the lamp. This heat sink can be disposed inside the housing/adapter as an independent structural unit. It is likewise feasible for this heat sink to serve as a heat-conducting means between the lamp and the wall of the housing so that the heat is dissipated toward the outside directly by way of the housing. This is particularly suitable when the housing is a metallic housing having good conduction of heat.

It should be noted at this point that the adapter comprising the lamp can be handled independently, and it is necessary to have a power supply unit for operating the adapter on the one side and a light guide for coupling the light on the other. Any desired applications can be derived from such an advantageous construction.

The power supply unit can be a stationary unit. However, for promoting mobility, it is advantageous if the power supply unit is accommodated in a separate and small housing as far as possible, preferably within the meaning of a storage battery pack. Accordingly, the housing of the power supply unit comprises a socket for inserting the plug of the housing or adapter comprising the lamp. The socket of the housing of the power supply unit and the plug of the housing of the lamp can be coordinated to each other in such a way that the full surface of the housing of the lamp bears against the housing of the power supply unit in the inserted state of the plug, which results in a stable structural unit that can be easily decoupled for replacing the power supply unit or the adapter. Thus a power supply unit having freshly charged storage batteries can be attached or an adapter having a different light source can be coupled at all times.

As in the case of the housing of the lamp, the socket can be integrated in the housing of the power supply unit. It is also feasible for the housing of the power supply unit to comprise a recess which is coordinated to the cross-section of the housing of the lamp and into which the housing of the lamp can be inserted at least slightly. Any desired mechanical and also magnetic coupling options between the two housings are feasible. It is also possible, in principle, to have an inductive power coupling in the case of an operation using alternating current.

There are different possibilities of implementing and developing the teaching of the present invention to advantage. For this purpose, reference should be made firstly to the subordinate claims and secondly to the following explanation of a preferred exemplary embodiment of the invention with reference to the drawings. Generally preferred design forms and developments of the teaching of this invention have also been described in conjunction with the explanation of the preferred exemplary embodiment of the invention with reference to the drawings, in which:

The single FIGURE is a diagrammatic and partial cutaway view of an exemplary embodiment of a light source of the invention comprising the suggested power supply unit.

The single FIGURE shows, in outlines, a light source of the invention for coupling light into a surgical handheld device (not shown in the FIGURE), more particularly a handheld device used in ophthalmology. The light source comprises a lamp 1, a light guide 2 extending from the lamp 1, and a power supply unit 3.

According to the invention, the lamp 1 is disposed in an adapter-like housing 4, the housing 4 or the adapter comprising a connection 5 for the power supply and a connection 6 for the light guide 2.

The single FIGURE further shows that the connection 5 is formed by a plug 7 that is integrated in the housing 4 and that protrudes from the housing 4. The plug 7 communicates with a corresponding socket 8 that is integrated in a housing 9 of the power supply unit 3. When the two housings 4, 9 are arranged and designed suitably, the electrical connection between the two can be established such that the adapter-like housing 4 bears directly against the housing 9 comprising the power supply unit 3. With appropriate coordination in terms of shape and size, the electrical coupling between the two housings and locking means enable a structural unit to be formed that can be detached again, if required.

A lamp 1 in the form of an LED is formed inside the adapter-like housing 4. The LED is held by a heat sink 10 that transports heat directly toward the exterior of the housing 4 or toward the housing wall.

An optical system 11 that together with an inserted light guide 2 or with a suitable coupling element 12 forms the coupling point 13 for coupling light is disposed downstream of the lamp 1. The coupling element 12 can be secured or locked in position inside the housing 4 by the use of suitable clamping means 14.

In order to avoid repetitions, reference should be made to the general portion of the description for additional features that are not revealed in the figures.

In conclusion, it should be pointed out that the above exemplary embodiment merely serves for explaining the light source of the invention by way of example without restricting it to said embodiment.

### LIST OF REFERENCE NUMERALS

1 Lamp
2 Light guide
3 Power supply unit
4 Adapter-like housing
5 Connection for the power supply unit
6 Connection for the light guide
7 Plug (electricity)
8 Socket (electricity)
9 Housing (for the power supply unit)
10 Heat sink
11 Optical system, lens
12 Coupling element
13 Coupling point
14 Clamping means, means for locking

1.-19. (canceled)

20. A light source for coupling light into a medical device, more particularly a surgical handheld device, preferably for use in ophthalmology, said light source comprising:
a lamp;
a light guide extending from the lamp; and
a power supply unit for the lamp,
wherein the lamp is disposed in an adapter-like housing comprising a connection for the power supply unit and a connection for the light guide.

21. The light source as defined in claim 20, wherein the housing is a one-piece component.

22. The light source as defined in claim 20, wherein the housing is in the form of a cylindrical sleeve.

23. The light source as defined in claim 20, wherein the connections for the power supply unit and the light guide respectively are disposed on opposite sides of the housing.

24. The light source as defined in claim 20, wherein the connection for the power supply unit is preferably in the form of a plug assigned to the housing.

25. The light source as defined in claim 24, wherein the plug protrudes from an end side of the housing.

26. The light source as defined in claim 20, wherein the connection for the light guide is in the form of a receptacle that is integrated in the housing and is intended for the coupling end of a light guide or a bundle of light guides.

27. The light source as defined in claim 26, wherein the receptacle is substantially defined by the inner wall of the housing.

28. The light source as defined in claim 26, wherein the receptacle comprises means for locking the light guide in its coupled position.

29. The light source as defined in claim 28, wherein the means are in the form of clamping means.

30. The light source as defined in claim 20, wherein the lamp is in the form of an LED or an array comprising a plurality of LEDs.

31. The light source as defined in claim 20, wherein a lens or a lens system is disposed downstream of the lamp inside the housing.

32. The light source as defined in claim 20, wherein a heat sink is assigned to the lamp.

33. The light source as defined in claim 20, wherein the power supply unit is accommodated in a separate housing.

34. The light source as defined in claim 33, wherein the housing comprises a socket for inserting a plug of the housing comprising the lamp.

35. The light source as defined in claim 34, wherein the socket is integrated in the housing of the power supply unit.

36. The light source as defined in claim 20, wherein the housings of the power supply unit and the lamp are approximately adapted to each other.

37. The light source as defined in claim 36, wherein the two housings result in one structural unit when connected together.

38. The light source as defined in claim 20, wherein the power supply unit comprises one or more storage batteries in its housing.
